SEQUENCE LISTING (16 sequences)

SEQ ID NO: 1 - protein, 459 aa, Mus musculus
MRVLILLWLFTAFPGILSDVQLQESGPGLVKPSQSLSLTCTVTGYSITSDYAWNWIRQFPGNKLEWMVYITYSGITGYNPSLKSRISITRDTSKNQFFLQLNSVTTGDTATYYCARYTTTAFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 2 - protein, 117 aa, Artificial sequence
  variable region in SEQ ID NO 1
DVQLQESGPGLVKPSQSLSLTCTVTGYSITSDYAWNWIRQFPGNKLEWMVYITYSGITGYNPSLKSRISITRDTSKNQFFLQLNSVTTGDTATYYCARYTTTAFDYWGQGTTLTVSS

SEQ ID NO: 3 - DNA, 1380 bp, Mus musculus
atgagagtgctgattcttttgtggctgttcacagcctttcctggtatcctgtctgatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggatggtctacataacctacagtggtatcactggctataacccctctctcaaaagtcggatctctatcactcgagacacatccaagaaccagttcttcctgcagttgaattctgtgactactggggacacagccacctattactgtgcaagatatactacgactgcgtttgactactggggccaaggcaccactctcacggtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 4 - DNA, 351 bp, Artificial sequence
  variable coding sequence in SEQ ID NO 3
gatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggatggtctacataacctacagtggtatcactggctataacccctctctcaaaagtcggatctctatcactcgagacacatccaagaaccagttcttcctgcagttgaattctgtgactactggggacacagccacctattactgtgcaagatatactacgactgcgtttgactactggggccaaggcaccactctcacggtctcctca

SEQ ID NO: 5 - protein, 238 aa, Mus musculus
MKLPVRLLVLMFWIPASRSDIVMTQTPLSLPVSLGDQASISCRSSQSLVHSNGKTYLHWYLQKPGQSPKLLIYKVSNRFSGVPDRFSGSGSGTDFTLKISRVEAEDLGVYFCSQSTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 6 - protein, 100 aa, Artificial sequence
  variable region in SEQ ID NO 5
DIVMTQTPLSLPVSLGDQASISCRSSQSLVHSNGKTYLHWYLQKPGQSPKLLIYKVSNRFSGVPDRFSGSGSGTDFTLKISRVEAEDLGVYFCSQSTHVP

SEQ ID NO: 7 - DNA, 717 bp, Mus musculus
atgaagttgcctgttaggctgttggtgctgatgttctggattcctgcttccaggagtgatattgtgatgacccaaactccactctccctgcctgtcagtcttggagatcaagcctccatctcttgcagatctagtcagagccttgtacacagtaatggaaaaacctatttacattggtacctgcagaagccaggccagtctcctaagctcctgatctacaaagtttccaaccgattttctggggtcccagacaggttcagtggcagtggatcagggacagatttcacactcaagatcagcagagtggaggctgaggatctgggagtttatttctgctctcaaagcacacatgttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 8 - DNA, 300 bp, Artificial sequence
  variable coding sequence in SEQ ID NO 7
gatattgtgatgacccaaactccactctccctgcctgtcagtcttggagatcaagcctccatctcttgcagatctagtcagagccttgtacacagtaatggaaaaacctatttacattggtacctgcagaagccaggccagtctcctaagctcctgatctacaaagtttccaaccgattttctggggtcccagacaggttcagtggcagtggatcagggacagatttcacactcaagatcagcagagtggaggctgaggatctgggagtttatttctgctctcaaagcacacatgttccg

SEQ ID NO: 9 - DNA, 1395 bp, Mus musculus
atgtacttgggactgaactatgtattcatagtttttctcttaaatggtgtccagagtgaattgaagcttgaggagtctggaggaggcttggtgcagcctggaggatccatgaaactctcttgtgctgcctctggattcacttttagtgacgcctggatggactgggtccgccagtctccagagaaggggcttgagtggattgctgaaattagaagcaaagctaataattatgcaacatactttgctgagtctgtgaaagggaggttcaccatctcaagagatgattccaaaagtggtgtctacctgcaaatgaacaacttaagagctgaggacactggcatttatttctgtaccaagttatcactacgttactggttcttcgatgtctggggcgcagggaccacggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 10 - protein, 464 aa, Mus musculus
MYLGLNYVFIVFLLNGVQSELKLEESGGGLVQPGGSMKLSCAASGFTFSDAWMDWVRQSPEKGLEWIAEIRSKANNYATYFAESVKGRFTISRDDSKSGVYLQMNNLRAEDTGIYFCTKLSLRYWFFDVWGAGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 11 - DNA, 363 bp, Artificial sequence
  variable coding sequence in SEQ ID NO 9
gaattgaagcttgaggagtctggaggaggcttggtgcagcctggaggatccatgaaactctcttgtgctgcctctggattcacttttagtgacgcctggatggactgggtccgccagtctccagagaaggggcttgagtggattgctgaaattagaagcaaagctaataattatgcaacatactttgctgagtctgtgaaagggaggttcaccatctcaagagatgattccaaaagtggtgtctacctgcaaatgaacaacttaagagctgaggacactggcatttatttctgtaccaagttatcactacgttactggttcttcgatgtctggggcgcagggaccacggtcaccgtctcctca

SEQ ID NO: 12 - protein, 121 aa, Artificial sequence
  variable region in SEQ ID NO 10
ELKLEESGGGLVQPGGSMKLSCAASGFTFSDAWMDWVRQSPEKGLEWIAEIRSKANNYATYFAESVKGRFTISRDDSKSGVYLQMNNLRAEDTGIYFCTKLSLRYWFFDVWGAGTTVTVSS

SEQ ID NO: 13 - DNA, 720 bp, Mus musculus
atgatgagtcctgcccagttcctgtttctgttagtgctctggattcgggaaaccaacggtgattttgtgatgacccagactccactcactttgtcggttaccattggacaaccagcctccatctcttgcaagtcaagtcagagcctcttggatagtgatggaaagacatatttgaattggttgttacagaggccaggccagtctccaaagcacctcatctatctggtgtctaaactggactctggagtccctgacaggttcactggcagtggatcagggaccgatttcacactgagaatcagcagagtggaggctgaggatttgggagtttattattgctggcaaagtacacattttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctacaagtggaagattgatggcagtgaacgacaaaatggcgtcctgaacagttggactgatcagcccaaagacatcaatgtcgacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 14 - protein, 239 aa, Mus musculus
MMSPAQFLFLLVLWIRETNGDFVMTQTPLTLSVTIGQPASISCKSSQSLLDSDGKTYLNWLLQRPGQSPKHLIYLVSKLDSGVPDRFTGSGSGTDFTLRISRVEAEDLGVYYCWQSTHFPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 15 - DNA, 339 bp, Artificial sequence
  variable coding sequence in SEQ ID NO 13
gattttgtgatgacccagactccactcactttgtcggttaccattggacaaccagcctccatctcttgcaagtcaagtcagagcctcttggatagtgatggaaagacatatttgaattggttgttacagaggccaggccagtctccaaagcacctcatctatctggtgtctaaactggactctggagtccctgacaggttcactggcagtggatcagggaccgatttcacactgagaatcagcagagtggaggctgaggatttgggagtttattattgctggcaaagtacacattttccgtggacgttcggtggaggcaccaagctggaaatcaaacgg

SEQ ID NO: 16 - protein, 113 aa, Artificial sequence
  variable region in SEQ ID NO 14
DFVMTQTPLTLSVTIGQPASISCKSSQSLLDSDGKTYLNWLLQRPGQSPKHLIYLVSKLDSGVPDRFTGSGSGTDFTLRISRVEAEDLGVYYCWQSTHFPWTFGGGTKLEIKR

(19) United States
(12) Patent Application Publication
(10) Pub. No.: US 2012/0034225 A1
(43) Pub. Date: Feb. 9, 2012

(54) Suppressing Bone Loss with Anti-IL-19 Antibody

(75) Inventor: MING-SHI CHANG, TAINAN (TW)

(73) Assignee: NATIONAL CHENG KUNG UNIVERSITY, TAINAN (TW)

(21) Appl. No.: 12/849,350

(22) Filed: Aug. 3, 2010

(51) Int. Cl. A61K 39/395; A61P 19/10; A61P 19/08

(52) U.S. Cl. 424/135.1; 424/158.1; 424/133.1; 424/145.1; 424/136.1

(57) ABSTRACT

A method of suppressing bone loss with an anti-IL-19 antibody, optionally in combination with an anti-IL-20 antibody or an anti-RANKL antibody.

### BACKGROUND OF THE INVENTION

Bones make up skeletons, which provide structure and support for bodies. They also serve as a storehouse for minerals such as calcium.

The body constantly breaks down old bones and builds up new bones. Net bone loss occurs when old bones are broken down faster than new bones are made. Bone loss is evident in osteoporosis and disorders associated with osteolysis (e.g., cancer and infection). Accompanied with pain and an increased risk of bone fracture, bone loss can significantly affect life quality.

It is of great importance to identify new agents for suppressing bone loss.

### SUMMARY OF THE INVENTION

The present invention is based on unexpected discoveries that an anti-IL-19 monoclonal antibody significantly inhibits osteoclast differentiation in vitro and suppresses bone loss in vivo.

Accordingly, one aspect of this invention features a method of suppressing bone loss in a subject in need thereof an effective amount of a composition containing an anti-IL-19 antibody (e.g., monoclonal antibody 1BB1 or a genetically engineered antibody derived from it), and optionally, an anti-IL-20 antibody (monoclonal antibody 7E or a genetically engineered antibody derived from it), an anti-RANKL antibody (antibody AM162), or both. In one example, the subject is a human patient suffering from osteoporosis, e.g., that associated with estrogen deficiency. In another example, he or she suffers from osteolysis caused by, e.g., cancer bone metastasis.

The anti-IL-19, anti-IL-20, or anti-RANKL antibody can be a naturally-occurring antibody (e.g., a monoclonal antibody), an antigen-binding fragment thereof (e.g., F(ab′)₂, Fab, or Fv), or a genetically engineered antibody (e.g., chimeric antibody, humanized antibody, or single-chain antibody) that neutralizes IL-19, IL-20, or RANKL, i.e., binding to one of these antigens and blocking the signaling pathway mediated by it.

The anti-IL-19 antibody can contain (1) a heavy chain variable region (V_{H}) that includes all of the complementarity-determining regions (CDRs) in the V_{H} of antibody 1BB 1 (SEQ ID NO:2), and (2) a light chain variable region (V_{L}) that includes all of the CDRs in the V_{L} of antibody 1BB1 (SEQ ID NO:6). In one example, this anti-IL-19 antibody contains the same V_{H} and V_{L} of 1BB1.

The anti-IL-20 antibody can contain (1) a V_{H} that includes all of the CDRs in the V_{H} of antibody 7E (SEQ ID NO:12), and (2) a V_{L} that includes all of the CDRs in the V_{L} of antibody 7E (SEQ ID NO:16). In one example, this anti-IL-20 antibody contains the same V_{H} and V_{L} of antibody 7E.

When the above-described composition contains two antibodies (i.e., an anti-IL-19 antibody and an anti-IL-20 or anti-RANKL antibody), these two antibodies can form a bi-specific complex. In one example, both of the antibodies are Fab fragments that form a bi-specific antibody.

Also within the scope of this invention are (1) a pharmaceutical composition for suppressing bone loss, the composition containing an anti-IL-19 antibody and, optionally, an anti-IL-20 or anti-RANKL antibody, and (2) the use of this composition in manufacturing a medicament for suppressing bone loss.

The details of one or more embodiments of the invention are set forth in the description below. Other features or advantages of the present invention will be apparent from the following drawings and detailed description of several examples, and also from the appended claims.

### BRIEF DESCRIPTION OF THE DRAWINGS

The drawings are first described.

FIG. 1 is a chart showing the effect of antibody 1BB1 in suppressing bone loss in CIA rats. The values shown in this figure are means ± standard deviations. *: P<0.05 as compared with saline-treated rats.

FIG. 2 is a chart showing the effect of antibody 1BB1 in inhibiting osteoclast differentiation in vitro from hematopoetic stem cells induced by macrophage colony-stimulating factor and RANKL. *: P<0.05 as compared with mIgG control. **: P<0.01 as compared with mIgG control.

FIG. 3 is a chart showing the effect of antibody 1BB1 in suppressing bone loss cased by breast cancer in mice. The values shown in this figure are means ± standard deviations. *: P<0.05 as compared with mIgG-treated mice.

### DETAILED DESCRIPTION OF THE INVENTION

We have discovered that anti-IL-19 antibody, unexpectedly, suppressed bone loss via, at least, inhibition of osteoclast differentiation.

Accordingly, the present invention relates to a method for suppressing bone loss in a subject in need thereof an effective amount of a pharmaceutical composition containing an anti-IL-19 antibody. The subject (e.g., a human patient) may suffer from oeteoporosis (e.g., that caused by estrogen deficiency) or osteolysis, the latter being evident in various diseases (e.g., neoplastic, infectious, metabolic, traumatic, vascular, congenital and articular disorders). Many types of cancer cells (e.g., breast cancer cells, prostate cancer cells, colon cancer cells, lung cancer cells, renal cell carcinoma cells, cells of giant cell tumor of bone, or multiple myeloma cells) can metastasize to the bone, leading to boss loss via osteolysis. Thus, a subject to be treated in the method of this invention can be a cancer patient who suffers from or is at risk for cancer bone metastasis.

As used herein, the term “an effective amount” refers to the amount of each active agent required to confer therapeutic effect on the subject, either alone or in combination with one or more other active agents. Effective amounts vary, as recognized by those skilled in the art, depending on route of administration, excipient choice, and co-usage with other active agents. The term “antibody” used herein refers to naturally-occurring immunoglobulins, antigen-binding fragments thereof, or generically engineered antibodies known in the art.

Naturally-occurring anti-IL-19 antibodies, either polyclonal or monoclonal, can be prepared by conventional methods, using an IL-19 protein or a fragment thereof as the inducing antigen. See, e.g., Harlow and Lane, (1988) Antibodies: A Laboratory Manual, Cold Spring Harbor Laboratory, New York. A “monoclonal antibody” refers to a homogenous antibody population and a “polyclonal antibody” refers to a heterogenous antibody population. These two terms do not limit the source of an antibody or the manner in which it is made. IL-19 is a cytokine well known in the art. For example, human IL-19 can be retrieved from the GenBank under accession numbers:

Human IL-19 isoform 1: NP_{—}715639 (protein) and NM_{—}153758.1 (gene)

Human IL-19 isoform 2: NP_{—}037503 (protein) and NM_{—}013371.2 (gene)

To produce an anti-IL-19 antibody, this protein or a fragment thereof can be coupled to a carrier protein, such as KLH, mixed with an adjuvant, and injected into a host animal. Antibodies produced in the animal can then be purified by a protein A column and/or affinity chromatography. Commonly employed host animals include rabbits, mice, guinea pigs, and rats. Various adjuvants that can be used to increase the immunological response depend on the host species and include Freund's adjuvant (complete and incomplete), mineral gels such as aluminum hydroxide, CpG, surface-active substances such as lysolecithin, pluronic polyols, polyanions, peptides, oil emulsions, keyhole limpet hemocyanin, and dinitrophenol. Useful human adjuvants include BCG (bacille Calmette-Guerin) and Corynebacterium parvum.

Polyclonal antibodies are present in the sera of the immunized subjects. Monoclonal antibodies can be prepared using standard hybridoma technology (see, for example, Kohler et al. (1975) Nature 256, 495; Kohler et al. (1976) Eur. J. Immunol. 6, 511; Kohler et al. (1976) Eur J Immunol 6, 292; and Hammerling et al. (1981) Monoclonal Antibodies and T Cell Hybridomas, Elsevier, N.Y.). In particular, monoclonal antibodies can be obtained by any technique that provides for the production of antibody molecules by continuous cell lines in culture such as described in Kohler et al. (1975) Nature 256, 495 and U.S. Pat. No. 4,376,110; the human B-cell hybridoma technique (Kosbor et al. (1983) Immunol Today 4, 72; Cole et al. (1983) Proc. Natl. Acad. Sci. USA 80, 2026, and the EBV-hybridoma technique (Cole et al. (1983) Monoclonal Antibodies and Cancer Therapy, Alan R. Liss, Inc., pp. 77-96). Such antibodies can be of any immunoglobulin class including IgG, IgM, IgE, IgA, IgD, and any subclass thereof. The hybridoma producing the monoclonal antibodies of the invention may be cultivated in vitro or in vivo. The ability to produce high titers of monoclonal antibodies in vivo makes it a particularly useful method of production. After obtaining antibodies specific to IL-19, their ability to neutralize IL-19 can be determined by a routine procedure.

Fully human anti-IL-19 antibodies, such as those expressed in transgenic animals are also features of the invention. See, e.g., Green et al., Nature Genetics 7:13 (1994), and U.S. Pat. Nos. 5,545,806 and 5,569,825.

Antigen-binding fragments (e.g., F(ab′)₂, Fab, or Fv) of a naturally-occurring antibody can be generated by known techniques. For example, F(ab′)₂ fragments can be produced by pepsin digestion of an antibody molecule and Fab fragments can be generated by reducing the disulfide bridges of F(ab′)₂ fragments.

The anti-IL-19 antibody to be used in this invention can also be a genetically engineered antibody, e.g., a humanized antibody, a chimeric antibody, a single chain antibody (scFv), or a domain antibody (dAb; see Ward, et. Al., 1989, Nature, 341:544-546). Such an antibody has substantially the same antigen-binding residues/regions as a naturally-occurring antibody from which it derives, thereby preserving the same antigen specificity as the naturally-occurring antibody.

A humanized antibody contains a human immunoglobulin (i.e., recipient antibody) in which regions/residues responsible for antigen binding (i.e., the CDRs, particularly the specific-determining residues therein) are replaced with those from a non-human immunoglobulin (i.e., donor antibody). In some instances, one or more residues inside a frame region of the recipient antibody are also replaced with those from the donor antibody. A humanized antibody may also contain residues from neither the recipient antibody nor the donor antibody. These residues are included to further refine and optimize antibody performance. Antibodies can also be humanized by methods known in the art, e.g., recombinant technology.

A chimeric antibody is a molecule in which different portions are derived from different animal species, such as those having a variable region derived from a murine monoclonal antibody and a human immunoglobulin constant region. Such an antibody can be prepared via routine techniques described in, e.g., Morrison et al. (1984) Proc. Natl. Acad. Sci. USA 81, 6851; Neuberger et al. (1984) Nature 312, 604; and Takeda et al. (1984) Nature 314:452.

A single-chain antibody can be prepared via recombinant technology by linking a nucleotide sequence coding for a V_{H} chain and a nucleotide sequence coding for a V_{L} chain. Preferably, a flexible linker is incorporated between the two variable regions. Alternatively, techniques described for the production of single chain antibodies (U.S. Pat. Nos. 4,946,778 and 4,704,692) can be adapted to produce a phage scFv library and scFv clones specific to IL-19 can be identified from the library following routine procedures. Positive clones can be subjected to further screening to identify those that suppress IL-19 activity.

In one example, the anti-IL-19 antibody to be used in the method of this invention is monoclonal antibody 1BB1 (see Hsing et al., Cytokine 44:221-228; 2008), an antigen binding fragment thereof, or a genetically-engineered functional variant thereof. Shown below are the amino acid sequences for the heavy and light chains of this monoclonal antibody, as well as their encoding nucleotide sequences:

Heavy chain amino acid sequence:
(SEQ ID NO: 1)
M R V L I L L W L F T A F P G I L S D V Q L Q E S G P G L V K P S Q S L S L T
C T V T G Y S I T S D Y A W N W I R Q F P G N K L E W M V Y I T Y S G I T G Y
N P S L K S R I S I T R D T S K N Q F F L Q L N S V T T G D T A T Y Y C A R Y
T T T A F D Y W G Q G T T L T V S S A K T T P P S V Y P L A P G S A A Q T N S
M V T L G C L V K G Y F P E P V T V T W N S G S L S S G V H T F P A V L Q S D
L Y T L S S S V T V P S S T W P S E T V T C N V A H P A S S T K V D K K I V P
R D C G C K P C I C T V P E V S S V F I F P P K P K D V L T I T L T P K V T C
V V V D I S K D D P E V Q F S W F V D D V E V H T A Q T Q P R E E Q F N S T F
R S V S E L P I M H Q D W L N G K E F K C R V N S A A F P A P I E K T I S K T
K G R P K A P Q V Y T I P P P K E Q M A K D K V S L T C M I T D F F P E D I T
V E W Q W N G Q P A E N Y K N T Q P I M D T D G S Y F V Y S K L N V Q K S N W
E A G N T F T C S V L H E G L H N H H T E K S L S H S P G K
Italic region: signal peptide
Bold-faced region: variable chain (SEQ ID NO: 2)
Bold-faced and underlined regions: CDRs
Regular font regions: constant regions
Underlined region: hinge region
Heavy chain nucleotide sequence:
(SEQ ID NO: 3)
ATGAGAGTGCTGATTCTTTTGTGGCTGTTCACAGCCTTTCCTGGTATCCTGTCTGATGTGCAGCTTCAGGAGTCGGGA
CCTGGCCTGGTGAAACCTTCTCAGTCTCTGTCCCTCACCTGCACTGTCACTGGCTACTCAATCACCAGTGATTATGCC
TGGAACTGGATCCGGCAGTTTCCAGGAAACAAACTGGAGTGGATGGTCTACATAACCTACAGTGGTATCACTGGCTAT
AACCCCTCTCTCAAAAGTCGGATCTCTATCACTCGAGACACATCCAAGAACCAGTTCTTCCTGCAGTTGAATTCTGTG
ACTACTGGGGACACAGCCACCTATTACTGTGCAAGATATACTACGACTGCGTTTGACTACTGGGGCCAAGGCACCACT
CTCACGGTCTCCTCAGCCAAAACGACACCCCCATCTGTCTATCCACTGGCCCCTGGATCTGCTGCCCAAACTAACTCC
ATGGTGACCCTGGGATGCCTGGTCAAGGGCTATTTCCCTGAGCCAGTGACAGTGACCTGGAACTCTGGATCCCTGTCC
AGCGGTGTGCACACCTTCCCAGCTGTCCTGCAGTCTGACCTCTACACTCTGAGCAGCTCAGTGACTGTCCCCTCCAGC
ACCTGGCCCAGCGAGACCGTCACCTGCAACGTTGCCCACCCGGCCAGCAGCACCAAGGTGGACAAGAAAATTGTGCCC
AGGGATTGTGGTTGTAAGCCTTGCATATGTACAGTCCCAGAAGTATCATCTGTCTTCATCTTCCCCCCAAAGCCCAAG
GATGTGCTCACCATTACTCTGACTCCTAAGGTCACGTGTGTTGTGGTAGACATCAGCAAGGATGATCCCGAGGTCCAG
TTCAGCTGGTTTGTAGATGATGTGGAGGTGCACACAGCTCAGACGCAACCCCGGGAGGAGCAGTTCAACAGCACTTTC
CGCTCAGTCAGTGAACTTCCCATCATGCACCAGGACTGGCTCAATGGCAAGGAGTTCAAATGCAGGGTCAACAGTGCA
GCTTTCCCTGCCCCCATCGAGAAAACCATCTCCAAAACCAAAGGCAGACCGAAGGCTCCACAGGTGTACACCATTCCA
CCTCCCAAGGAGCAGATGGCCAAGGATAAAGTCAGTCTGACCTGCATGATAACAGACTTCTTCCCTGAAGACATTACT
GTGGAGTGGCAGTGGAATGGGCAGCCAGCGGAGAACTACAAGAACACTCAGCCCATCATGGACACAGATGGCTCTTAC
TTCGTCTACAGCAAGCTCAATGTGCAGAAGAGCAACTGGGAGGCAGGAAATACTTTCACCTGCTCTGTGTTACATGAG
GGCCTGCACAACCACCATACTGAGAAGAGCCTCTCCCACTCTCCTGGTAAATGA
Italic region: signal peptide coding sequence
Bold-faced region: variable chain coding sequence (SEQ ID NO: 4)
Bold-faced and underlined regions: CDR coding sequences
Regular font regions: constant region coding sequences
Underlined region: hinge region coding Sequence
Light chain amino acid sequence:
(SEQ ID NO: 5)
M K L P V R L L V L M F W I P A S R S D I V M T Q T P L S L P V S L G D Q A S
I S C R S S Q S L V H S N G K T Y L H W Y L Q K P G Q S P K L L I Y K V S N R
F S G V P D R F S G S G S G T D F T L K I S R V E A E D L G V Y F C S Q S T H
V P W T F G G G T K L E I K R A D A A P T V S I F P P S S E Q L T S G G A S V
V C F L N N F Y P K D I N V K W K I D G S E R Q N G V L N S W T D Q D S K D S
T Y S M S S T L T L T K D E Y E R H N S Y T C E A T H K T S T S P I V K S F N
R N E C
Italic region: signal peptide
Bold-faced region: variable chain (SEQ ID NO: 6)
Bold-faced and underlined regions: CDRs
Regular font region: constant region
Underlined region: joining segment
Light chain nucleotide sequence:
(SEQ ID NO: 7)
ATGAAGTTGCCTGTTAGGCTGTTGGTGCTGATGTTCTGGATTCCTGCTTCCAGGAGTGATATTGTGATGACCCAAACT
CCACTCTCCCTGCCTGTCAGTCTTGGAGATCAAGCCTCCATCTCTTGCAGATCTAGTCAGAGCCTTGTACACAGTAAT
GGAAAAACCTATTTACATTGGTACCTGCAGAAGCCAGGCCAGTCTCCTAAGCTCCTGATCTACAAAGTTTCCAACCGA
TTTTCTGGGGTCCCAGACAGGTTCAGTGGCAGTGGATCAGGGACAGATTTCACACTCAAGATCAGCAGAGTGGAGGCT
GAGGATCTGGGAGTTTATTTCTGCTCTCAAAGCACACATGTTCCGTGGACGTTCGGTGGAGGCACCAAGCTGGAAATC
AAACGGGCTGATGCTGCACCAACTGTATCCATCTTCCCACCATCCAGTGAGCAGTTAACATCTGGAGGTGCCTCAGTC
GTGTGCTTCTTGAACAACTTCTACCCCAAAGACATCAATGTCAAGTGGAAGATTGATGGCAGTGAACGACAAAATGGC
GTCCTGAACAGTTGGACTGATCAGGACAGCAAAGACAGCACCTACAGCATGAGCAGCACCCTCACGTTGACCAAGGAC
GAGTATGAACGACATAACAGCTATACCTGTGAGGCCACTCACAAGACATCAACTTCACCCATTGTCAAGAGCTTCAAC
AGGAATGAGTGTTAG
Italic region: signal peptide coding sequence
Bold-faced region: variable chain coding sequence (SEQ ID NO: 8)
Bold-faced and underlined regions: CDR coding sequences
Regular font region: constant region coding sequence
Underlined region: joining segment coding sequence

Antibody 1BB 1 can be produced by a conventional method, i.e., produced from a hybridoma cell line as described in Hsing et al., Cytokine 44:221-228; 2008, synthesized chemically, or expressed via recombinant technology.

A functional variant of 1BB1 contains a V_{H} at least 75% (80%, 85%, 90%, or 95%) identical to that of 1BB1 (SEQ ID NO:2) and a V_{L} at least 75% (80%, 85%, 90%, or 95%) identical to that of 1BB 1 (SEQ ID NO:6). As used herein, “percent homology” of two amino acid sequences is determined using the algorism described in Karlin and Altschul, Proc, Natl. Acad. Sci. USA 87:2264-2268, 1990, modified as described in Karlin and Altschul, Proc, Natl. Acad. Sci. USA 5873-5877, 1993. Such an algorism is incorporated into the NBLAST and XBLAST programs of Altschul et al., J. Mol. Biol. 215:403-410, 1990. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al., Nucleic Acids Res. 25:3389-3402, 1997. When utilizing the BLAST and Gapped BLAST programs, the default parameters of the respective programs (e.g., XBLAST and NBLAST) are used. See www.ncbi.nlm.nih.gov.

A functional variant of 1BB1 (e.g., a humanized antibody) can be generated by introducing mutations in a frame region (FR) of either the V_{H} or V_{L} of 1BB 1 and keep intact their CDRs, particularly the specific-determining residues in these regions. It is well known that CDRs of an antibody determine its specificity. Accordingly, mutations in FRs normally would not affect antibody specificity. The CDRs and FRs of an antibody can be determined based on the amino acid sequences of its V_{H} and V_{L}. See www.bioinf.org.uk/abs. The binding-specificity of the functional equivalents described herein can be examined using methods known in the art, e.g., ELISA or Western-blot analysis.

Alternatively, a functional variant of 1BB 1 is a genetically engineered antibody containing the same V_{H} and V_{L} as 1BB1. Such a variant (e.g., a chimeric antibody or a single-chain antibody) can be prepared following methods described above.

If necessary, any of the anti-IL-19 antibodies can be co-used with an anti-IL-20 antibody or an anti-RANKL antibody. Anti-IL-20 or anti-RANKL antibodies can be prepared by any of the methods described above, using IL-20, RANKL, or a fragment thereof as the inducing antigen. IL-20 is a member of the IL-10 cytokine family. Human IL-20 is described under GenBank Accession Number NP_{—}061194 (protein) and NM_{—}018724 (gene). RANKL (Receptor Activator for Nuclear Factor κ B Ligand), also known as TNF-related activation-induced cytokine (TRANCE), osteoprotegerin ligand (OPGL), and ODF (osteoclast differentiation factor), is a protein molecule important in bone metabolism. Human RANKL is described under GenBank Accession Number AAB86811 (protein) and AF019047 (gene).

In one example, monoclonal antibody 7E, which neutralizes IL-20 activity, or a functional variant thereof, is co-used with an anti-IL-19 antibody for suppressing bone loss. mAb7E is produced by the hybridoma cell line deposited at the American Type Culture Collection, 10801 University Boulevard, Manassas, Va. 20110-2209, U.S.A. and assigned a deposit number PTA-8687. See U.S. Pat. No. 7,435,800 and US 20090048432. This hybridoma cell line will be released to the public irrevocably and without restriction/condition upon granting a US Patent on this application, and will be maintained in the ATCC for a period of at least 30 years from the date of the deposit for the enforceable life of the patent or for a period of 5 years after the date of the most recent. The amino acid sequences/cDNA sequences of the heavy and light chains of mAb7E are shown below.

| Nucleotide sequence (SEQ ID NO: 9) and amino acid sequence | |
|---|---|
| (SEQ ID NO: 10) of mAb 7E heavy chain | |
| atg tac ttg gga ctg aac tat gta ttc ata gtt ttt ctc tta aat | |
| M Y L G L N Y V F I V F L L N | 15 |
| ggt gtc cag agt gaa ttg aag ctt gag gag tct gga gga ggc ttg | |
| G V Q S E L K L E E S G G G L | 30 |
| gtg cag cct gga gga tcc atg aaa ctc tct tgt gct gcc tct gga | |
| V Q P G G S M K L S C A A S G | 45 |
| ttc act ttt agt gac gcc tgg atg gac tgg gtc cgc cag tct cca | |
| F T F S D A W M D W V R Q S P | 60 |
| gag aag ggg ctt gag tgg att gct gaa att aga agc aaa gct aat | |
| E K G L E W I A E I R S K A N | 75 |
| aat tat gca aca tac ttt gct gag tct gtg aaa ggg agg ttc acc | |
| N Y A T Y F A E S V K G R F T | 90 |
| atc tca aga gat gat tcc aaa agt ggt gtc tac ctg caa atg aac | |
| I S R D D S K S G V Y L Q M N | 105 |
| aac tta aga gct gag gac act ggc att tat ttc tgt acc aag tta | |
| N L R A E D T G I Y F C T K L | 120 |
| tca cta cgt tac tgg ttc ttc gat gtc tgg ggc gca ggg acc acg | |
| S L R Y W F F D V W G A G T T | 135 |
| gtc acc gtc tcc tca gcc aaa acg aca ccc cca tct gtc tat cca | |
| V T V S S A K T T P P S V Y P | 150 |
| ctg gcc cct gga tct gct gcc caa act aac tcc atg gtg acc ctg | |
| L A P G S A A Q T N S M V T L | 165 |
| gga tgc ctg gtc aag ggc tat ttc cct gag cca gtg aca gtg acc | |
| G C L V K G Y F P E P V T V T | 180 |
| tgg aac tct gga tcc ctg tcc agc ggt gtg cac acc ttc cca gct | |
| W N S G S L S S G V H T F P A | 195 |
| gtc ctg cag tct gac ctc tac act ctg agc agc tca gtg act gtc | |
| V L Q S D L Y T L S S S V T V | 210 |
| ccc tcc agc acc tgg ccc agc gag acc gtc acc tgc aac gtt gcc | |
| P S S T W P S E T V T C N V A | 225 |
| cac ccg gcc agc agc acc aag gtg gac aag aaa att gtg ccc agg | |
| H P A S S T K V D K K I V P R | 240 |
| gat tgt ggt tgt aag cct tgc ata tgt aca gtc cca gaa gta tca | |
| D C G C K P C I C T V P E V S | 255 |
| tct gtc ttc atc ttc ccc cca aag ccc aag gat gtg ctc acc att | |
| S V F I F P P K P K D V L T I | 270 |
| act ctg act cct aag gtc acg tgt gtt gtg gta gac atc agc aag | |
| T L T P K V T C V V V D I S K | 285 |
| gat gat ccc gag gtc cag ttc agc tgg ttt gta gat gat gtg gag | |
| D D P E V Q F S W F V D D V E | 300 |
| gtg cac aca gct cag acg caa ccc cgg gag gag cag ttc aac agc | |
| V H T A Q T Q P R E E Q F N S | 315 |
| act ttc cgc tca gtc agt gaa ctt ccc atc atg cac cag gac tgg | |
| T F R S V S E L P I M H Q D W | 330 |
| ctc aat ggc aag gag ttc aaa tgc agg gtc aac agt gca gct ttc | |
| L N G K E F K C R V N S A A F | 345 |
| cct gcc ccc atc gag aaa acc atc tcc aaa acc aaa ggc aga ccg | |
| P A P I E K T I S K T K G R P | 360 |
| aag gct cca cag gtg tac acc att cca cct ccc aag gag cag atg | |
| K A P Q V Y T I P P P K E Q M | 375 |
| gcc aag gat aaa gtc agt ctg acc tgc atg ata aca gac ttc ttc | |
| A K D K V S L T C M I T D F F | 390 |
| cct gaa gac att act gtg gag tgg cag tgg aat ggg cag cca gcg | |
| P E D I T V E W Q W N G Q P A | 405 |
| gag aac tac aag aac act cag ccc atc atg gac aca gat ggc tct | |
| E N Y K N T Q P I M D T D G S | 420 |
| tac ttc gtc tac agc aag ctc aat gtg cag aag agc aac tgg gag | |
| Y F V Y S K L N V Q K S N W E | 435 |
| gca gga aat act ttc acc tgc tct gtg tta cat gag ggc ctg cac | |
| A G N T F T C S V L H E G L H | 450 |
| aac cac cat act gag aag agc ctc tcc cac tct cct ggt aaa TGA | |
| N H H T E K S L S H S P G K — | 464 |

The bold-faced region refers to the V_{H} of mAb 7E heavy chain (DNA sequence SEQ ID NO: 11; protein sequence SEQ ID NO: 12)

| Nucleotide sequence (SEQ ID NO: 13) and amino acid sequence (SEQ | |
|---|---|
| ID NO: 14) of mAb 7E light chain | |
| atg atg agt cct gcc cag ttc ctg ttt ctg tta gtg ctc tgg att | |
| M M S P A Q F L F L L V L W I | 15 |
| cgg gaa acc aac ggt gat ttt gtg atg acc cag act cca ctc act | |
| R E T N G D F V M T Q T P L T | 30 |
| ttg tcg gtt acc att gga caa cca gcc tcc atc tct tgc aag tca | |
| L S V T I G Q P A S I S C K S | 45 |
| agt cag agc ctc ttg gat agt gat gga aag aca tat ttg aat tgg | |
| S Q S L L D S D G K T Y L N W | 60 |
| ttg tta cag agg cca ggc cag tct cca aag cac ctc atc tat ctg | |
| L L Q R P G Q S P K H L I Y L | 75 |
| gtg tct aaa ctg gac tct gga gtc cct gac agg ttc act ggc agt | |
| V S K L D S G V P D R F T G S | 90 |
| gga tca ggg acc gat ttc aca ctg aga atc agc aga gtg gag gct | |
| G S G T D F T L R I S R V E A | 105 |
| gag gat ttg gga gtt tat tat tgc tgg caa agt aca cat ttt ccg | |
| E D L G V Y Y C W Q S T H F P | 120 |
| tgg acg ttc ggt gga ggc acc aag ctg gaa atc aaa cgg gct gat | |
| W T F G G G T K L E I K R A D | 135 |
| gct gca cca act gta tcc atc ttc cca cca tcc agt gag cag tta | |
| A A P T V S I F P P S S E Q L | 150 |
| aca tct gga ggt gcc tca gtc gtg tgc ttc ttg aac aac ttc tac | |
| T S G G A S V V C F L N N F Y | 175 |
| aag tgg aag att gat ggc agt gaa cga caa aat ggc gtc ctg aac | |
| P K D I N V K W K I D G S E R | 180 |
| agt tgg act gat cag ccc aaa gac atc aat gtc gac agc aaa gac | |
| Q N G V L N S W T D Q D S K D | 195 |
| agc acc tac agc atg agc agc acc ctc acg ttg acc aag gac gag | |
| S T Y S M S S T L T L T K D E | 210 |
| tat gaa cga cat aac agc tat acc tgt gag gcc act cac aag aca | |
| Y E R H N S Y T C E A T H K T | 225 |
| tca act tca ccc att gtc aag agc ttc aac agg aat gag tgt tag | |
| S T S P I V K S F N R N E C — | 239 |

The bold-faced region refers to the V_{L} of mAb 7E light chain (DNA sequence SEQ ID NO: 15; protein sequence SEQ ID NO: 16).

When two antibodies are used in suppressing bone loss, they can form a bi-specific complex (i.e., bi-specific antibody), which contains two antigen-binding domains (i.e., two heavy-light chain pairs), one specific to IL-19 and the other specific to IL-20 or RANKL. Such a bi-specific antibody can be prepared via conventional methods.

To suppress bone loss, any of the anti-IL-19 antibodies described herein can be mixed with a pharmaceutically acceptable carrier, either alone or in combination with an anti-IL-20 or anti-RANKL antibody, to form a pharmaceutical composition. “Acceptable” means that the carrier must be compatible with the active ingredient of the composition (and preferably, capable of stabilizing the active ingredient) and not deleterious to the subject to be treated. Suitable carriers include microcrystalline cellulose, mannitol, glucose, defatted milk powder, polyvinylpyrrolidone, and starch, or a combination thereof.

The above-described pharmaceutical composition can be administered via a conventional route, e.g., orally, parenterally, by inhalation spray, topically, rectally, nasally, buccally, vaginally or via an implanted reservoir, to suppressing bone loss. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques.

A sterile injectable composition, e.g., a sterile injectable aqueous or oleaginous suspension, can be formulated according to techniques known in the art using suitable dispersing or wetting agents (such as Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

In addition, the pharmaceutical composition described above can be administered to the subject via injectable depot routes of administration such as using 1-, 3-, or 6-month depot injectable or biodegradable materials and methods.

Without further elaboration, it is believed that one skilled in the art can, based on the above description, utilize the present invention to its fullest extent. The following specific embodiments are, therefore, to be construed as merely illustrative, and not limitative of the 2 5 remainder of the disclosure in any way whatsoever. All publications cited herein are incorporated by reference.

### Example 1

### Suppressing Bone Loss in CIA Rats by Antibody 1BB 1

Arthritis, which results in bone loss, was induced in Sprague-Dawley rats (8-week old) by bovine type II collagen as follows. SD rats were immunized initially by intradermal injection (in the dorsum) of 200 μl emulsion containing Freund's complete adjuvant, 4 mg/ml heat-killed Mycobacterium tuberculosis (Arthrogen-CIA; Chondrex, Redmond, Wash.), and bovine type II collagen (CII; 2 mg/ml dissolved in 0.05 M acetic acid) at a ratio of 1:1:1 (v/v/v). On day 8, the rats were injected subcutaneously with 100 μl of the just-described emulsion in the roots of the tails to boost their immune responses. CIA was observed in these rats between day 11 and day 13 after the initial immunization.

The following three groups of rats (n=7) were subjected to this study:

Group (1): healthy rats

Group (2): CIA rats administered with PBS (s.c.) 10 days after the first injection of type II collagen, and

Group (3): CIA rats administered with antibody 1BB1 (20 mg/kg, s.c.) 10 days after the first injection of type II collagen.

Microcomputed tomographic analysis, using a 1076 microCT-40 system (Skyscan, Aartselaar, Belgium) equipped with a high resolution, low-dose X-ray scanner, was performed to assess the efficacy of 1BB1 in protecting bone destruction in CIA rats. The X-ray tube in the scanner was operated with photon energy of 48 kV, current of 200 uA, and exposure time of 1180 ms through a 0.5-mm-thick filter. The image pixel size was 17.20 um, and the scanning time was approximately 15 min. After standardized reconstruction of the scanned images, the data sets for each tibia sample were resampled with software (CTAn; Skyscan) to orient each sample in the same manner. Consistent conditions such as thresholds were applied throughout all analyses. Bone mineral density, a three-dimensional bone characteristic parameter, was analyzed in 50 consecutive slices. The results were calculated as a percentage versus values relative to a PBS control.

The tibias obtained from the CIA rats treated with PBS showed prominent bone damage compared to the intact joints found in healthy rats. The CIA rats treated with 1BB 1 displayed alleviated bone loss as compared to the rats treated with PBS.

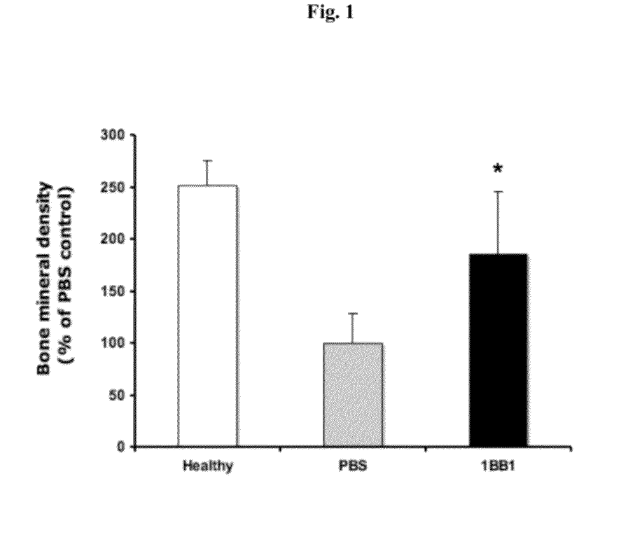

The bone mineral density, a quantitative parameter for assessing disease severity, was measured in each treated CIA rat as described above. 1BB1 successfully suppressed bone loss in CIA rats as compared to PBS (P<0.05). See FIG. 1.

### Example 2

### Inhibiting Osteoclast Differentiation by Antibody 1BB 1

Bone marrow cells (BMCs) were isolated from the tibias of C57BL6 mice and incubated for 12 h at 37° C. with 5% CO₂ in a a-MEM medium. Non-adherent cells were collected and placed in a 24-well plate (2×10⁶ cells per well) and cultured in the same medium supplemented with 30 ng/ml recombinant murine macrophage colony-stimulating factor (M-CSF) (PeproTech) for 48 hours to induce BMC differentiation into osteoclast precursor cells. The precursor cells thus obtained were then treated with anti-IL-19 monoclonal antibody 1BB1 at various concentrations (2-6 mg/ml) or a control mouse IgG (mIgG) at a concentration of 6 μg/ml. Both antibody 1BB1 and mIgG were dissolved in α-MEM supplemented with M-CSF (40 ng/ml) and sRANKL (100 ng/ml) (PeproTech). The culture medium was changed every 3 days. Eight days later, the cells were collected and fixed in acetone and the number of the osteoclasts in them were determined by Tartrate-resistant Acid Phosphatase (TRAP) staining, using an acid phosphatase kit (Sigma-Aldrich).

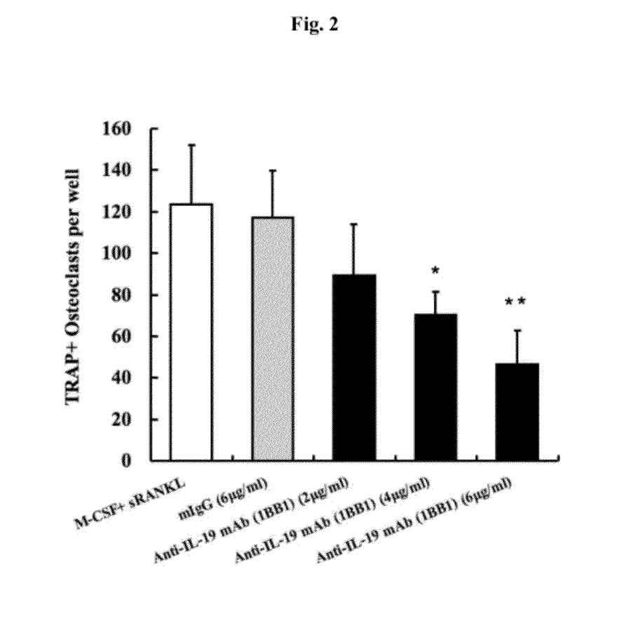

As shown in FIG. 2, antibody 1BB1 significantly inhibited osteoclast differentiation in a dose-dependent manner as compared to the mIgG control. This suggests that anti-IL-19 antibody is effective in blocking bone resorption mediated by osteoclast.

### Example 3

### Suppressing Bone Loss Caused by Breast Cancer Cells

Mouse breast cancer 4T1 cells, at a concentration of 2×10^{5/}100 μL, were injected directly into the left ventricle of 6-wk-old female BALB/c mice, which were anesthetized with pentobarbital (Sigma-Aldrich) at 50 mg/kg body weight via i.p., using an insulin syringe (29 gauge, BD Ultra-Fine; Becton Dickinson). After injection, the mice were randomly assigned into 3 groups (n=6/group), each treated by i.p. as follows:

Group 1: treated with PBS as a vehicle control three time in one week

Group 2: treated with a control mouse IgG (mIgG) at 10 mg/kg three times in one week

Group 3: treated with anti-IL-19 antibody 1BB1 at 10 mg/kg three times in one week.

Mice not injected with the cancer cells were used as healthy controls.

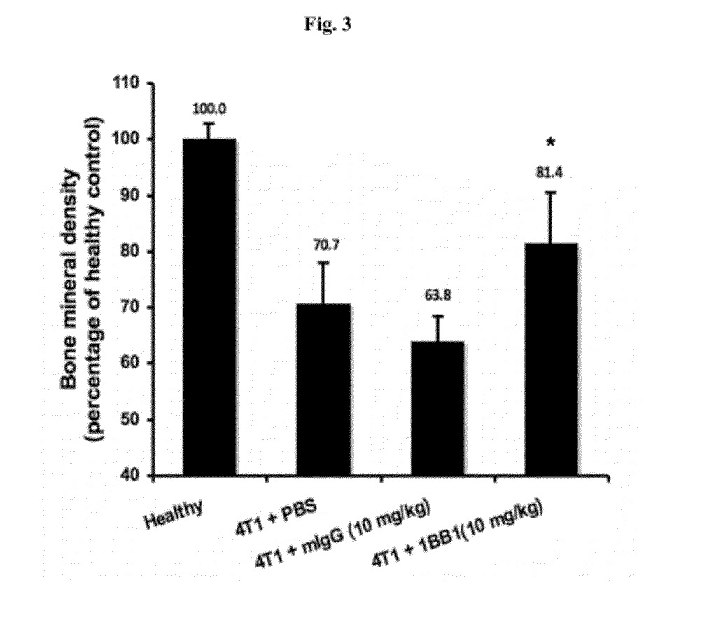

Twenty days post treatment, the tibia metaphyses of the mice were analyzed in-vivo on a micro-CT (1076; SkyScan) with a high resolution, low-dose X-ray scanner. Bone mineral density (BMD), a three-dimensional bone characteristic parameter, was analyzed in 50 consecutive slices. The results thus obtained were shown in FIG. 3. The Y axis values were calculated by the formula: (BMD of treated mice/BMD of healthy controls) %. The BMD of the mice injected with the cancer cells were reduced as compared to that of healthy control mice. This cancer-induced reduction of BMD was rescued significantly by antibody 1BB1.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method for suppressing bone loss, the method comprising administering to a subject in need thereof an effective amount of a composition containing an anti-IL-19 antibody.

2. The method of claim 1, wherein the anti-IL-19 antibody is a humanized, antibody, a chimeric antibody, a single-chain antibody, a naturally-occurring antibody or an antigen-binding fragment thereof.

3. The method of claim 2, wherein the anti-IL-19 antibody contains a heavy chain variable region including all of the complementarity-determining regions in SEQ ID NO:2 and a light chain variable region including all of the complementarity-determining regions in SEQ ID NO:6.

4. The method of claim 3, wherein the anti-IL-19 antibody contains a heavy chain variable region including SEQ ID NO:2 and a light chain variable region including SEQ ID NO:6.

5. The method of claim 4, wherein the anti-IL-19 antibody is a chimeric antibody or a single-chain antibody.

6. The method of claim 4, wherein the anti-IL-19 antibody is monoclonal antibody 1BB1 or an antigen-binding fragment thereof.

7. The method of claim 1, wherein the composition further contains an anti-IL-20 antibody, an anti-RANKL antibody, or both.

8. The method of claim 7, wherein the composition contains an anti-IL-20 antibody that forms a bi-specific complex with the anti-IL-19 antibody.

9. The method of claim 8, wherein both the anti-IL-19 antibody and the anti-IL-20 antibody are Fab fragments.

10. The method of claim 8, wherein the anti-IL-19 antibody contains a heavy chain variable region including all of the complementarity-determining regions in SEQ ID NO:2 and a light chain variable region including all of the complementarity-determining regions in SEQ ID NO:6 and the anti-IL-20 antibody contains a heavy chain variable region including all of the complementarity-determining regions in SEQ ID NO:12 and a light chain variable region including all of the complementarity-determining regions in SEQ ID NO:16.

11. The method of claim 10, wherein the anti-IL-19 antibody is a Fab fragment of monoclonal antibody 1BB1 and the anti-IL-20 antibody is a Fab fragment of monoclonal antibody 7E.

12. The method of claim 7, wherein the composition contains an anti-RANKL antibody that forms a bi-specific complex with the anti-IL-19 antibody.

13. The method of claim 12, wherein the anti-IL-19 antibody contains a heavy chain variable region including all of the complementarity-determining regions in SEQ ID NO:2 and a light chain variable region including all of the complementarity-determining regions in SEQ ID NO:6.

14. The method of claim 12, wherein both the anti-IL-19 antibody and the anti-RANKL antibody are Fab fragments.

15. The method of claim 14, wherein the anti-IL-19 antibody is a Fab fragment of monoclonal antibody 1BB 1 and the anti-RANKL antibody is a Fab fragment of antibody AMG 162.

16. The method of claim 1, wherein the subject is a human patient suffering from osteoporosis.

17. The method of claim 16, wherein the anti-IL-19 antibody is a humanized antibody, a chimeric antibody, a single-chain antibody, a naturally-occurring antibody or an antigen-binding fragment thereof.

18. The method of claim 17, wherein the anti-IL-19 antibody contains a heavy chain variable region including all of the complementarity-determining regions in SEQ ID NO:2 and a light chain variable region including all of the complementarity-determining regions in SEQ ID NO:6.

19. The method of claim 18, wherein the anti-IL-19 antibody is antibody 1BB1 or an antigen-binding fragment thereof.

20. The method of claim 16, wherein the composition further contains an anti-IL-20 antibody, an anti-RANKL antibody, or both.

21. The method of claim 20, wherein the anti-IL-20 antibody or the anti-RANKL antibody forms a bi-specific complex with the anti-IL-19 antibody.

22. The method of claim 1, wherein the subject is a human patient suffering from osteolysis induced by a cancer.

23. The method of claim 22, wherein the cancer is breast cancer, prostate cancer, colon cancer, lung cancer, renal cell carcinoma, giant cell tumor of bone, or multiple myeloma.

24. The method of claim 22, wherein the anti-IL-19 antibody is a humanized antibody, a chimeric antibody, a single-chain antibody, a naturally-occurring antibody or an antigen-binding fragment thereof.

25. The method of claim 24, wherein the anti-IL-19 antibody contains a heavy chain variable region including all of the complementarity-determining regions in SEQ ID NO:2 and a light chain variable region including all of the complementarity-determining regions in SEQ ID NO:6.

26. The method of claim 25, wherein the anti-IL-19 antibody is antibody 1BB 1 or an antigen-binding fragment thereof.

27. The method of claim 22, wherein the composition further contains an anti-IL-20 antibody, an anti-RANKL antibody, or both.

28. The method of claim 27, wherein the anti-IL-20 antibody or the anti-RANKL antibody forms a bi-specific complex with the anti-IL-19 antibody.
